(19) United States
(12) Patent Application Publication
(10) Pub. No.: US 2007/0219343 A1
(43) Pub. Date: Sep. 20, 2007

(54) Isobenzoxazinones And Their Use As Ultraviolet Light Absorbers

(75) Inventor: Christoph Kroehnke, Breisach-Oberrimsingen (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT INTERNATIONAL LTD., Muttenz 1 (CH)

(21) Appl. No.: 11/628,350

(22) PCT Filed: May 23, 2005
(86) PCT No.: PCT/IB05/01699

§ 371 Date: Dec. 1, 2006

(30) Foreign Application Priority Data
Jun. 3, 2004 (EP) 04013140.1

(51) Int. Cl. C07D 265/22

(52) U.S. Cl. 528/367; 528/425; 544/73

(57) ABSTRACT

The present invention relates to new substituted isobenzoxazinones, in particular of formula (I) as given in claim 1, and their use as ultraviolet light absorbers, in particular for organic polymers.

The present invention relates to new isobenzoxazinones, in particular of formula (I), and their use as ultraviolet light absorbers for organic materials, in particular for organic polymers.

The usability and lifetime of plastic articles is influenced by numerous parameters such as mechanical properties, density, molar mass and mass distribution of the polymer. Depending on the final use and the local conditions (temperature, stress, and environmental influences) during the service time, a lifetime of up to several decades must be guaranteed which can only be reached with the appropriate stabilizers and stabilizer combinations. Their contribution can be determined under accelerated test conditions. As important technical criteria optical properties like color are measured, for example as yellowness index (YI), and are used to assess the stabilization of polymer articles.

Effects in the polymer matrix e.g. change in cristallinity, free volume, morphology and coefficient of diffusion and reaction kinetics cause changes in the degradation mechanism.

One of the decisive criteria preventing rapid degradation of polymer articles by light is the use of UV absorbers in combination with appropriate lightstabilizers.

UV absorbers help to prevent polymer degradation by reducing the harmful effects of ultraviolet rays by absorption through chromophores and by lowering the initiation rate through deactivation of excited states in the polymer matrix. Generally UV absorbers are able to dissipate the energy of absorbed photons within the polymer matrix in a harmless way, e.g. as heat.

There are other mechanistic effects also discussed in terms of chemical transformation of hydroperoxides by means of UV absorbers without generation of free peroxi radicals by photolytical cleavage (as described in the chapter “Lightstabilizers” of F. Gugumus “Plastics additives handbook”, p. 206ff, editor H. Zweifel, Hanser Publishers, Munich (2001)).

Among all absorbers used so far for the protection of polymeric matrices it is distinguished between phenol type and non-phenolic UV absorbers providing photostability in the 300-400 nm wavelength range.

The phenol type absorbers act by the excited-state intramolecular proton transfer mechanism. For the non-phenolic UV absorbers the formation of charge-separated species after photoexcitation is considered as the mode of action.

A more detailed description of individual classes of UV absorbers and action modes is given in J. E. Pickett, titled “Permanence of UV absorbers in Plastics and Coatings”, Technical Report 97CRD170/General Electric, December 1997.

The majority of the UV absorbers, particularly the benzophenone derivatives, are efficient in the UV-A (from 320 to 400 nm) and UV-B (from 290 to 320 nm) range.

Parameters such as long term maintenance of properties, including appearance and in particular color, gloss and transparency, are highly important characteristics of technical polymers. Therefore, any improvement of such parameters can be regarded as a measurable innovation step. There is still a need to improve properties compared to known UV absorbers like hydroxybenzophenones, hydroxybenzotriazols, hydroxytriazines and unsubstituted benzoxazinones.

Unsubstituted benzoxazinones are known as versatile UV absorbers (U.S. Pat. No. 3,989,698 and U.S. Pat. No. 4,446,262) and are commercially available, e.g. 2,2′-p-phenylene-bis-(3,1,-benzoxazin-4-one) as represented below is available as Cyasorb® 3638 form Cytec.

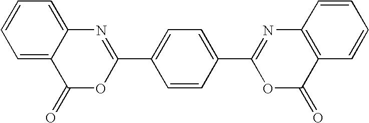

An improved process for the preparation of this compound and its use for transparent polymer materials is disclosed in WO 03/035735.

WO 03/016292 discloses substituted benzoxazinone compounds of the following formulae, as well as their use as ultraviolet light absorbers, in particular for organic polymers

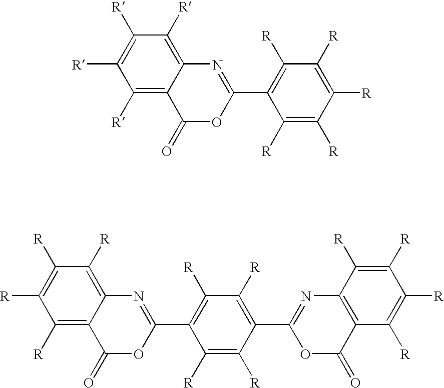

In EP 1 302 197 A1 and EP 1 317 918 A1 it is further disclosed that benzoxazinones, in particular 2,2′-p-phenylene-bis-(3,1-benzoxazin-4-one) are suitable as UV absorbers in cosmetic preparations such as sunscreens.

EP 0 674 038 A1 discloses the use of 4H-3,1-benzoxazin-4-one compounds for improving the lightfastness of textile materials. In particular 2,2′-p-phenylene-bis-(3,1-benzoxazin-4-one) is disclosed.

Surprisingly is has been found that substituted isobenzoxazinones as of the present invention are particularly useful as ultraviolet light absorbers which in terms of relevant technical parameters outperform the state of the art.

The present invention therefore relates to new substituted isobenzoxazinones of formula (I) 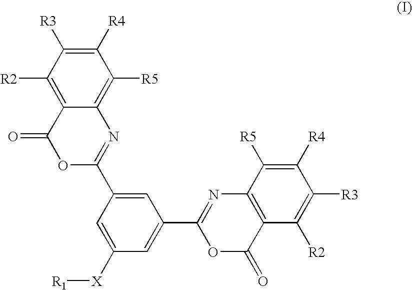
wherein
R₁ represents a long chain alkyl substituent, cycloalkyl substituent or isoalkyl substituent with CₙH₂ₙ₊₁ and n=8-30,
X represents a direct bond, —O—, an ester group (—COO—) or —S—.
R₂, R₃, R₄ and R₅ independently represent a substituent selected from hydrogen, halogen (F, Cl, Br, I) or C₁₋₁₂ alkyl.

In a preferred aspect the invention relates to new isobenzoxazinones of formula (I) wherein
R₁ represents a long chain alkyl or isoalkyl substituent with CₙH₂ₙ₊₁ and n=8-18,
X represents —O— and
R₂, R₃, R₄ and R₅ independently represent a substituent selected from hydrogen, fluorine, chlorine, bromine or C₁₋₄ alkyl.

More preferably R₂, R₃, R₄ and R₅ independently represent a substituent selected from hydrogen, fluorine, chlorine, methyl, ethyl, n-propyl, iso-propyl or tert.-butyl.

Most preferred compounds are those represented by formula (Ia) to (Ig):

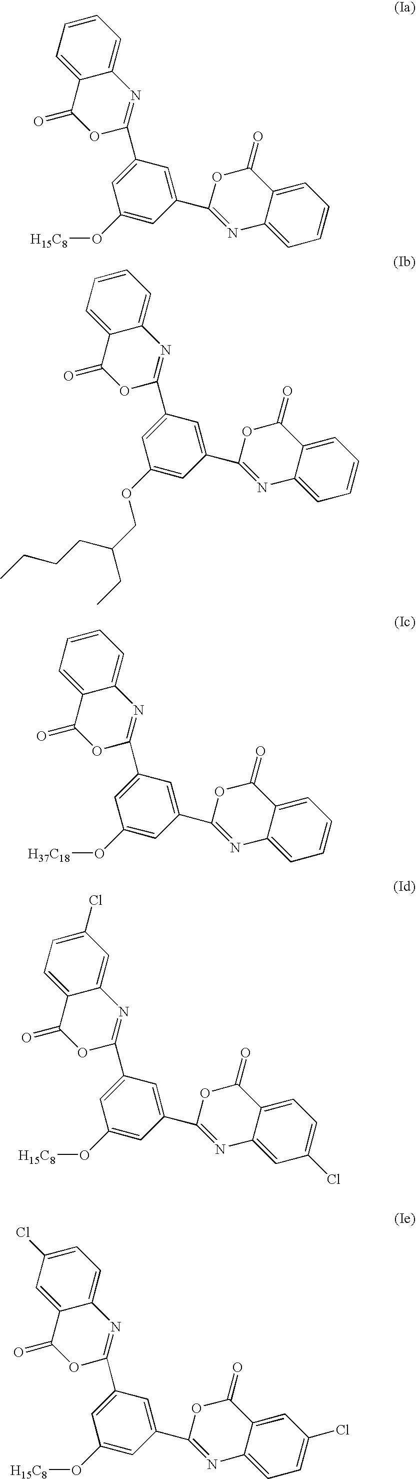

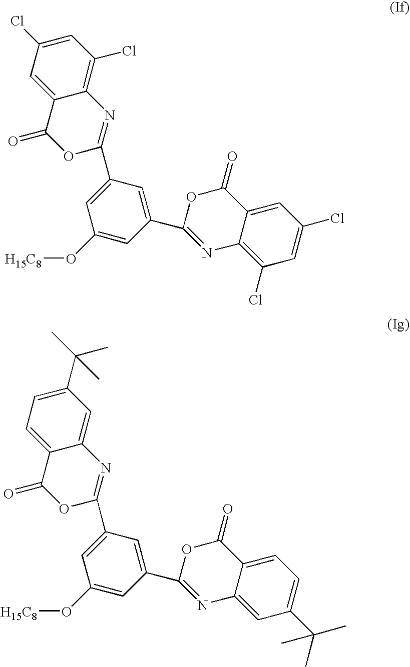

Moreover it was surprisingly found that particularly preferred compounds of formula (I) with X being —O— and R₁ being an unbranched alkyl substituent CₙH₂ₙ₊₁ with n=8-12 show morphological phase transitions below the corresponding melting points.

The new compounds according to the invention provide excellent stability against damage by light and oxidation and therefore are able to protect polymeric substrates against deterioration by degrading environmental influences.

Another object of the invention is the use of the new compounds as highly compatible UV absorbers in a large variety of organic substrates. In comparison to compounds known in the art, the new compounds according to the invention are by far better soluble in many organic substrates.

The new compounds according to the invention are preferably used in organic substrates in concentrations from 0.005 to 0.100 weight percent, most preferably in organic polymers.

The new compounds according to the invention are in particular suitable for organic polymers selected from the group of so called engineering plastics. Preferred organic polymers from the group of so called engineering plastics are polycarbonate, polyester and polyamide, in particular Polycarbonate (PC), Polyethyleneterephthalate (PET), Polyamide-6 (PA6) and Polyamide-6.6 (PA6.6).

Moreover, depending on their substitution pattern several of the new compounds can be also used in polyolefin based thermoplastic polymers.

Furthermore, the new compounds can be intercalated into clay type nanocomposites, a class of layered silicates which itself received considerable attention in fundamental research and industrial exploitation. Nanoclays provide enhanced properties already at very low filler content, usually below 5 wt-% including further improved thermal and oxidative stability of the intercalated compounds as for example described in M. Alexandre and P. Dubois, Polym. Mater. Sci. Eng, 28, 1-63 (2000), H. Quin, C. Zhao, S. Zhang, G. Chen and M. Yang, Polym. Degrad. Stab. 81, 497-500 (2003).

The new compounds are particularly suitable for cosmetic applications.

Preparation of the Compounds

The compounds according to the invention can be obtained, for example, by etherifying 3-hydroxyisophthalic acid according to the Williamson procedure with halogenated alkanes and preparing successively the final compounds.

The last step is the reaction of the corresponding diacid chlorides with anthranilic acid and anthranilic acid derivatives forming the isobenzoxazinone moieties. The preparation of corresponding isobenzoxazinones starting from hydroxyphthalic acid can be basically carried out according to the same procedure.

Suitable halogenated alkanes are for example 1-bromooctane, 1-bromononane, 1-bromodecane, 1-bromoundecane and higher homologues but also corresponding haloalkanes with branched or cyclic structures like 1-bromo-2-ethylhexane or bromocyclooctane, 3-bromocyclooctene, 2-(6-bromohexyloxy)-tetrahydro-2H-pyran etc. The haloalkane can also contain a saturated heterocycle, preferentially an oxygen containing heterocycle as represented for example by a compound like 2-(2-bromoethoxy)tetrahydro-2H-pyran, 2-(2-bromoethyl)-1,3-dioxane, 2-(2-bromoethyl)-2,5,5-trimethyl-1,3-dioxane etc.

Suitable starting materials are furthermore haloalkyl substituted arenes and aromatic heterocycles such as 3-(2-bromoethyl)indole, furthermore dibromosubstituted unbranched, branched and cyclic alkanes leading finally to dimeric structures. In the haloalkanes chlorine substitution can be used instead of bromine substitution.

Anthranilic (or 2-aminobenzoic) acid can be used for the final step as well as its derivatives like alkyl, dialkyl, trialkyl and tetraalkyl derivatives of the anthranilic acid linked at the 3-6 positions.

The alkyl, dialkyl, trialkyl and tetraalkyl substituents can have chain length from C₁₋₂₀ with linear, branched and cyclic structures, as well as 2-amino-3-chloro-benzoic acid, 2-amino-4-chloro-benzoic acid, 2-amino-5-chloro-benzoic acid, 2-amino-6-chloro-benzoic acid, 2-amino-3,4-dichloro-benzoic acid, 2-amino-3,5-dichloro-benzoic acid, 2-amino-3,6-dichloro-benzoic acid and further homologues. The halogen substitution can be fluorine or bromine instead of chlorine, as well as nitro- or cyano substitution.

### EXAMPLES

The following compounds were prepared by the method described below:

TABLE 1
Compounds related to the general Formula (I)

| Compound | Empirical | melting | | ε/mol⁻¹cm⁻¹ |
|---|---|---|---|---|
| No. | Formula | point/° C. | λ_{max.} | (solvent DMSO) |
| (Ia) | C₃₀H₂₈N₂O₅ | 174 | 288 | 34500 |
| (Ib) | C₃₀H₂₈N₂O₅ | 157 | 302 | 26600 |
| (Ic) | C₄₀H₄₈N₂O₅ | 151-153 | 283 | 17600 |
| (Id) | C₃₀H₂₆Cl₂N₂O₅ | 218 | 307 | 25500 |
| (Ie) | C₃₀H₂₆Cl₂N₂O₅ | 195 | 308 | 27600 |
| (If) | C₃₀H₂₄Cl₄N₂O₅ | 198 | 314 | 27400 |
| (Ig) | C₃₈H₄₄N₂O₅ | 192-193 | 304 | 24700 |

Said compounds can be obtained, for example, by a multistep synthesis starting from 5-hydroxyisophthalic acid (1). (Reference e.g. G. J. Bodwell, J. N. Bridson, M. K. Cyranski, J. W. J. Kennedy, T. M. Krygowski, M. R. Mannion and D. O. Miller, J. Org. Chem, 68 (6), 2089-2098 (2003)) 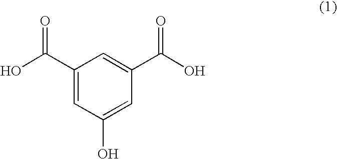
which is first converted under standard conditions and nearly quantitative yields into its dimethylester (2).

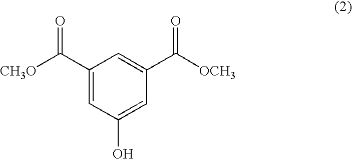

The synthesis towards the final compound (Ia), for example was continued by converting compound (2) with i-bromooctane according to the following procedure: 84.1 g (0.40 mol) compound (2) and 81.1 g (0.42 g) 1-bromooctane were dissolved in 250 ml dimethylformamide (DMF). Afterwards 61 g (0.44 mol) potassiumcarbonate was added. The slightly yellow dispersion was heated at T=120° during 4 hours. After cooling the solid precipitate was removed by filtration and washed with cold DMF. To the filtrate a total amount of 500 ml demineralized water was added under stirring and a precipitate was formed gradually. This precipitate was filtered and thoroughly washed with demineralized water until a neutral pH was reached and no further bromide ions could be detected using silvemitrate for detection purposes. After drying the product in the presence of phosphorouspentoxide in a desiccator under vacuum 127.0 g (98.6% yield) of a solid product (3) has been obtained.

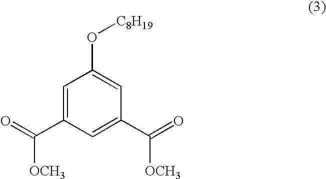

96,6 g (0.3 mol) of compound (3) has been hydrolysed by adding this product into 500 ml ethanol. After heating at a temperature of T=65° C. an aqueous solution of sodiumhydroxide (30 weight-%) has been added slowly during 90 minutes. The basic solution has been neutralised under stirring by means of an aqueous solution of hydrogenchloride (35 wt-%). Under those conditions the diacid (4) precipitated.

This product was separated by filtration, washed with demineralized water and dried in a vacuum oven at T=95° C. It remained 82.9 g (94% of theory) of a slightly yellow product, after further drying in a desiccator over phosphorus pentoxide.

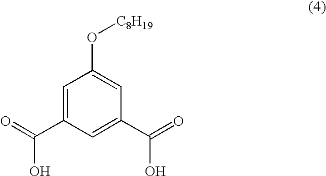

The diacid (4) was further converted into the diacid chloride (5) by adding in a vessel under nitrogen 150 ml of thionyl chloride and 82.0 g (0.279 mol) of product (4) and 1.5 ml dimethylformamide. This reaction mixture was first heated up to T=50° C. under reflux and under an increasing formation of a gas. Than the mixture was furthermore heated up to 75° C. After 3 hours the heating medium was removed and the excess of thionyl chloride was removed by distillation. The remaining product (5) precipitated partially. The solid product was isolated by filtration and washed with cold, dry ethanol; yield 73 g (79.7% of theory).

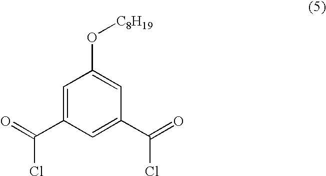

The final conversion towards product (Ia) took place by adding 540 ml water in a reaction vessel, together with 61.5 g (0.44 mol) of anthranilic acid (content 98 wg-%). Than 23.85 g (0.225 mol) of sodium carbonate has been included under stirring under slow formation of a solution (solution I).

73 g (0.22 mol) of product (5) was dissolved in 460 ml of acetone (solution II). This solution (II) was during 2.5 h added to the aqueous solution (I) with the anthranilic acid, under intensive stirring. A slightly exothermic reaction took place. The reaction mixture was than heated during 2 hours under reflux. By cooling a pale beige precipitate was formed which was filtered off at ambient temperatures, washed with water ad dried in the vacuum; it remained 117.3 g of a beige powder. This product was suspended in 350 ml of acetic anhydride and heated for 3 hours to reflux forming the final product which precipitated gradually. Onto addition of 20 ml of acetone the reaction mixture was filtered and thoroughly washed with acetone. After drying in the vacuum yielded 82.3 g (75.5% of th.) of the nearly colorless final product (Ia).

Application Tests in Ethylene/Methacrylic Anion Copolymers

An ethylene/methacrylic anion copolymer (“Ionomer”, containing a Zn cation), of the type Surlyn® 9910 (available from Du Pont) has been used for processing and long term exposure. 100 parts of this polymer have been mixed with 0.1 parts of the corresponding UV absorber. The composition of the formulations for examples 1 to 6 is given in table 2.

TABLE 2
UV absorbers used for processing of
ethylene/methacrylic acid copolymer

| Exam- | Content | | |
|---|---|---|---|
| ple | [%] | Name | Description of UV absorber |
| 1 | 0.10 | Tinuvin ® 327 | commercial benztriazole type |
| 2 | 0.10 | Tinuvin ® 328 | commercial benztriazole type |
| 3 | 0.10 | Cyasorb ® 1164 | commercial triazine type |
| 4 | 0.10 | Cyasorb ® 3638 | commercial benzoxazinone type |
| 5 | 0.10 | Compound (I) | iso benzoxazinone type |
| | | with R₁—X— = H | |
| 6 | 0.10 | Compound (Ia) | iso benzoxazinone type |

Examples 1-5 are comparative examples.

Pre-extrusion was carried out on single screw extruder type T30 (producer Collin) (screw type NS, screw composition 1:3, die diameter=4mm), at T=210° C. and a screw speed of 80 rpm. Afterwards Ionomer plaques (100×100×1 mm) were processed at a temperature of T=210° C. and a pressure of max. 100 bar during 24.6 seconds cycle time by means of an injection molding machine T 18 (producer Arburg), screw speed 400 rpm.

Accelerated thermal treatment was carried out by placing the injection molded plaques of the ionomer in an air circulated oven at T=60° C.

Yellowness Index (YI) was measured using a Minolta 3500d spectrocolorimeter mode CRIELL according to DIN 6167. Transparency was measured with the same instrument by measuring at a wavelength λ=700 nm. Finally gloss was determined at an angle of 60° using a glossmeter (type Byk).

The results after an exposure period of 24 hours are given in table 3.

TABLE 3
Oven ageing of ethylene/methacrylic acid copolymer
plaques containing different UV Absorbers

| Example | YI | % Transparency | Gloss/60° |
|---|---|---|---|
| 1 | 3.77 | 90.26 | 132.6 |
| 2 | 3.31 | 90.09 | 132.4 |
| 3 | 3.32 | 90.19 | 132.5 |
| 4 | 4.91 | 87.87 | 84.5 |
| 5 | 3.73 | 89.71 | 119.2 |
| 6 | 3.19 | 90.04 | 133.4 |

As the results of this oven tests it can be stated that:
the color in terms of Yellowness Index (YI) of the Ionomer plaques containing the novel compound (Ia) outperforms the commercial state-of-the-art UV absorbers of examples 1 to 3, and in particular the benzoxazinone of example 4.
the parameters like transparency and gloss are similar to those of the Ionomer plaques containing either benzotriazole or triazine based UV-absorbers.
in comparison with a conventional benzoxazinone type UV absorber and a corresponding unsubstituted isobenzoxazinone the substituted isobenzoxazinone according to the invention clearly show a superior performance.

Application Tests in Polycarbonate

Polycarbonate of the type Lexan® 141 (available from GE Plastics) has been used for processing and long term exposure. Prior to use the polymer was 4 hours dried at T=80° C. in the vacuum. 100 parts of this polymer have been mixed with 0.1 parts of the corresponding UV absorber. The composition of the formulations for examples 7 to 9 is given in table 4.

TABLE 4
UV absorbers used for processing and
long term exposure of polycarbonate

| Exam- | Content | | |
|---|---|---|---|
| ple | [%] | Name | Description of UV absorber |
| 7 | 0.10 | Tinuvin ® 234 | commercial benztriazole |
| | | | type |
| 8 | 0.10 | Hostavin ® B-CAP ® | commercial benzylidene- |
| | | | bis-malonate |
| 9 | 0.10 | Compound (Ic) | iso benzoxazinone type |
| 10 | 0.10 | Compound (Ig) | iso benzoxazinone type |

Examples 7 and 8 are comparative examples.

Mixing of the granular polymer and the granular additives took place by dryblending in a polyethylene bag. Pre-extrusion was carried out on single screw extruder type T30 (producer Collin) (screw type NS, L/D=20, screw composition 1:3, die diameter 4 mm), at T=260° C. (zone 1) up to 280° C. (zone 4) and a screw speed of 80 rpm. Afterwards polycarbonate plaques (thicknesses 1 mm) were processed at a temperature of T=265° C. to T=275° C. and a pressure of max. 100 bar at 20 seconds cycle time by means of an injection molding machine T 18 (producer Arburg), screw speed 400 rpm.

Accelerated thermal treatment (oven aging) was carried out by placing the injection molded plaques in an air circulated oven at T=120° C.

Transparency was measured with a spectrocolorimeter type Minolta CM 3500d by measuring at a wavelength λ=700 nm.

The results after an exposure period of up to 10 days are given in table 5.

TABLE 5
Oven ageing of polycarbonate (PC) plaques containing
different UV-Absorbers at T = 120° C.,
the values are % Transparency

| | Exposure time/days | | |
|---|---|---|---|
| Example | 2 | 5 | 10 |
| no UVA (virgin) | 90.11 | 90.06 | 90.07 |
| 7 | 90.16 | 90.07 | 90.03 |
| 8 | 90.03 | 89.89 | 90.00 |
| 9 | 90.25 | 90.13 | 90.12 |
| 10 | 90.22 | 90.11 | 90.11 |

As the results of this oven test in polycarbonate it can be stated that:
the transparency of the polycarbonate plaques with isobenzoxazinones is slightly superior in comparison to samples containing either benzotriazole or benzyliden-bis-malonate based UV-absorbers.

Application Tests in Polyethyleneterephthalate (PET)

Polyethyleneterephthalate (PET) of the type Arnite® D04 300 Natural (available from DSM) has been used for processing and long term exposure. Prior to use the polymer was 8 hours dried at T=120° C. in the vacuum. 100 parts of this polymer have been mixed with 0.2 parts of the phosphite type stabilizer Hostanox® PAR 24 and 0.05 parts of the phenol type stabilizer Hostanox® O 16 (the two latter stabilisers are used as base stabilisation and are commercially available from Clariant) and additionally with 0.25 parts of an UV absorber.

The composition of the formulations for examples 10 to 16 are given in table 6.

TABLE 6
UV absorbers used for processing and long term
exposure of polyethyleneterephthalate (PET)

| Exam- | Content | | |
|---|---|---|---|
| ple | [%] | Name | Description of UV absorber |
| 11¹⁾ | — | — | |
| 12 | 0.25 | Hostavin ® B-CAP ® | commercial benzylidene- |
| | | | bis-malonate |
| 13 | 0.25 | Sanduvor ® VSU ® | commercial oxanilide type |
| 14 | 0.25 | Nylostab ® S-EED ® | commercial multifunctional |
| | | | bis- HA(L)S terminated |
| | | | m-phenylene-bis-amide type |
| 15 | 0.25 | Cyasorb ® 3638 | commercial benzoxazinone |
| | | | type |
| 16 | 0.25 | Compound (Ib) | iso benzoxazinone type |
| 17 | 0.25 | Compound (Ig) | iso benzoxazinone type |

¹⁾only base stabilisation: 0.2 parts Hostanox ® PAR24, 0.05 parts Hostanox ® O 16 (see above).

Examples 11-14 and 16 are comparative examples.

Mixing of the granular polymer and the granular additives took place by dryblending in a polyethylene bag. Pre-extrusion was carried out on single screw extruder type T30 (producer Collin) (screw composition 1:3, die diameter=2 mm), at T=275° C. (zone 1) up to 285° C. (zone 5) and a screw speed of 80 rpm.

Afterwards polyethyleneterephthalate plaques (thickness 1 mm) were processed at temperatures of T=270-275° C. and a pressure of max. 85 bar (holding pressure 30 bar) at 28 seconds cycle time by means of an injection molding machine T 18 (producer Arburg), screw speed 300 rpm.

Accelerated thermal treatment (oven ageing) was carried out by placing the injection molded plaques in an air circulated oven at T=80° C.

Artificial exposure UV-A took place in an exposure device type Atlas UV-CON equipped with 8 fluorescent tubes emitting light of the wavelength λ=340 nm (according to ASTN D5208 cycle A) alternating over a period of 20 hours at T=50° C. and in the dark (4 hours) at a temperature of 40° C. (condensation phase).

The Yellowness Index (YI) was measured with a spectrocolorimeter type Minolta CM 3500d according to DIN 6167. Gloss measurements took place using a glossmeter type BYK.

The results after an exposure period of up to 15 days in the oven at T=80° C. are given in table 7, whereas the results of artificial exposure type UV-A are presented in table 8.

TABLE 7
Oven ageing of polyethyleneterephthalate (PET) plaques
containing different UV-Absorbers at T = 80° C.;
impact on Yellowness Index (YI)

| Example | YI |
|---|---|
| 11 | 2.41 |
| 12 | 2.32 |
| 13 | 3.90 |
| 14 | 4.32 |
| 15 | 2.35 |
| 16 | 2.10 |
| 17 | 2.21 |

TABLE 8
Artificial exposure UV-A of polyethyleneterephthalate
(PET) plaques containing different UV-Absorbers; impact
on gloss at 85° after 1500 hrs exposure time

| Example | Gloss (85°) |
|---|---|
| 11 | 92.8 |
| 12 | 88.8 |
| 13 | 91.8 |
| 14 | 87.1 |
| 15 | 86.4 |
| 16 | 94.8 |
| 17 | 95.5 |

As the results of this oven test with polyethyleneterephthalate (PET) plaques it can be stated that:
by heat treatment the color in terms of Yellowness Index (YI) containing the novel compound (Ia) and (Ig) outperforms the commercial state-of-the-art UV absorbers of the benzylidene-bis-malonate and oxanilide type.
the parameter like gloss with the new isobenzoxazinones (Ia) and (Ig) are superior to those of the polyethyleneterephthalate plaques containing either benzylidene-bis-malonate or oxanilide or conventional benzoxazinone based UV-absorbers.

Application Tests in Polyamide-6.6 (PA6.6)

Polyamide-6.6 (PA6.6) of the type PA6.6 Frianyl® A63E (available from Frisetta) has been used for processing and long term exposure. Prior to use the polymer was 8 hours dried at T=80° C. in the vacuum.

100 parts of this polymer (except formulation 18) have been mixed with 0.1 part of the phosphonite type stabiliser Sandostab® P-EPQ (used as base stabiliser) and additionally with 0.05-0.25parts of various lightstabilisers (mixtures or single components). The composition of the formulations for examples 18 to 21 are given in table 9.

TABLE 9
Stabilisers used for processing and long
term exposure of polyamide-6.6 (PA-6.6)

| Exam- | Content | | |
|---|---|---|---|
| ple | [%] | Name | Description of UV absorber |
| 18²⁾ | — | — | |
| 19³⁾ | 0.20 | Sanduvor ® VSU ® | commercial oxanilide type |
| 20³⁾ | 0.20 | Sanduvor ® VSU ® | commercial oxanilide type |
| | 0.05 | Nylostab ® S-EED ® | commercial multifunctional |
| | | | bis- HA(L)S terminated |
| | | | m-phenylene-bis-amide type |
| 21³⁾ | 0.10 | Sanduvor ® VSU ® | commercial oxanilide type |
| | 0.05 | Nylostab ® S-EED ® | commercial multifunctional |
| | | | bis- HA(L)S terminated |
| | | | m-phenylene-bis-amide type |
| | 0.05 | Compound I(a) | iso benzoxazinone type |

²⁾no stabiliser added (virgin formulation);
³⁾contains additionally 0.1 part Sandostab ® P-EPQ;

Examples 18-20 are comparative examples.

Mixing of the granular polymer and the granular additives took place by dryblending in a polyethylene bag. Pre-extrusion was carried out on single screw extruder type T30 (producer Collin) (screw composition 1:3, die diameter=2mm), at T=265° C. (zone 1) up to 275° C. (zone 5) and a screw speed of 60 rpm.

Afterwards polyamide-6.6 plaques (thickness 1 mm) were processed at a temperature of T=285° C. and a pressure of max. 80 bar (holding pressure 50 bar) at 19 seconds cycle time by means of an injection molding machine T 18 (producer Arburg), screw speed 300 rpm.

The plaques have been exposed by means of a Weather-O-Meter (according to D 4892 or ISO 11341-C): Xenon light, light continuous, dry conditions, light intensity 0.47 W/m² (at 340 nm), black panel temperature 63° C., relative humidity 50%, borosilicate S filter. The Yellowness Index (YI) was measured with a spectrocolorimeter type Minolta CM 3500d according to ISO 11341. Gloss measurements took place using a glossmeter type BYK.

The results for the gloss after an exposure period of 1250 hrs and for the yellowness index (YI) respectively for ΔE after 4500 hrs exposure are shown in table 10.

TABLE 10
Impact of Artificial Weathering (CAM-7) on color and gloss/85° of
various different polyamide-6.6 plaques (thickness 1 mm)

| | ΔE | YI | Gloss (85°) |
|---|---|---|---|
| Example | (after 4500 hrs) | (after 4500 hrs) | (after 1250 hrs) |
| 18 | 22.85 | 2.62 | 21.80 |
| 19 | 9.68 | −0.40 | 70.10 |
| 20 | 17.69 | −15.30 | 82.00 |
| 21 | 8.77 | −16.27 | 89.90 |

As the results of this test with polyamide-6.6 plaques it can be stated that:
formulations containing iso benzoxazinones can improve the performance with regard to color and gloss.

1. Isobenzoxazinones of formula (I) 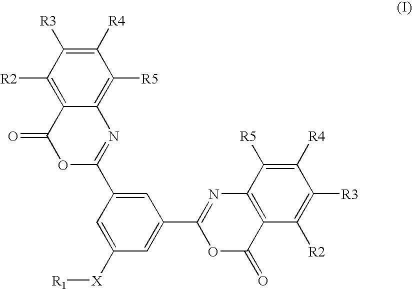
wherein
R₁ is a long chain alkyl substituent, cycloalkyl substituent or isoalkyl substituent with CₙH₂ₙ₊₁ and n=8-30
X is a direct bond, —O—, an ester group (—COO—) or —S—.
R₂, R₃, R₄ and R₅ independently are a substituent selected from the group consisting of hydrogen, halogen and C₁₋₁₂ alkyl.

2. Isobenzoxazinones according to claim 1, wherein
R₁ is a long chain alkyl or isoalkyl substituent with CₙH₂ₙ₊₁ and n=8-18,
X is —O— and
R₂, R₃, R₄ and R₅ independently are a substituent selected from the group consisting of hydrogen, fluoro, chloro and C₁₋₄ alkyl.

3. Isobenzoxazinones according to claim 1, wherein
R₁ is a long chain alkyl or isoalkyl substituent with CₙH₂ₙ₊₁ and n=8-18,
X is —O— and
R₂, R₃, R₄ and R₅ independently are a substituent selected from the group consisting of hydrogen, fluoro, chloro methyl, ethyl, n-propyl, iso-propyl and tert.-butyl.

4. Isobenzoxazinones according to claim 1, wherein the compound of formula (I) is a compound of the formula (Ia) to (Ig):

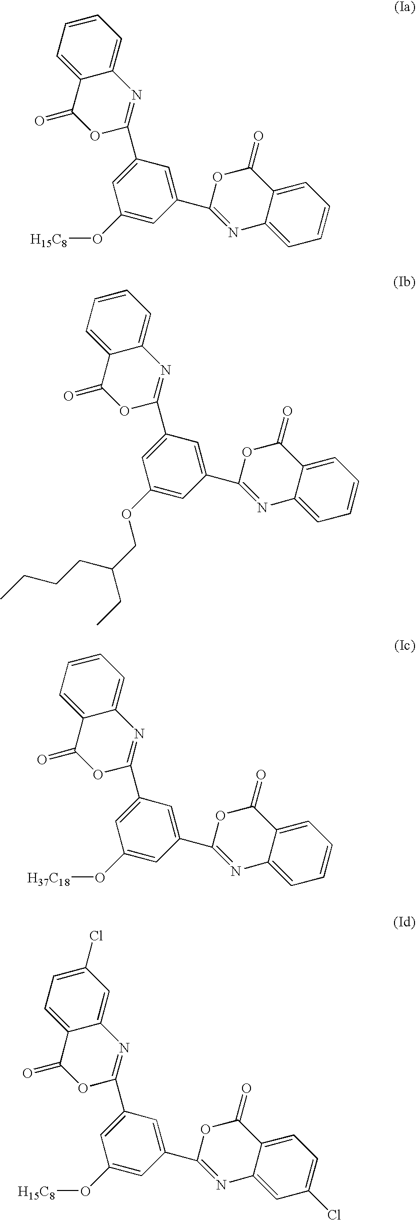

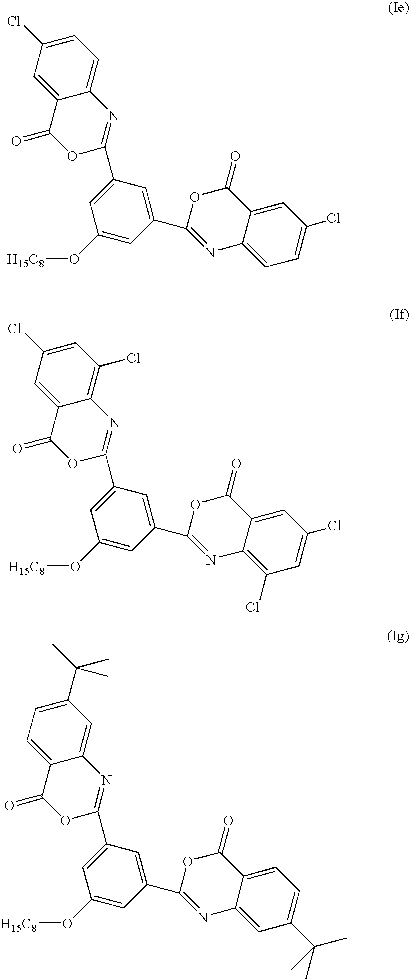

5. A process for the preparation of isobenzoxazinones according to claim 1, comprising the steps of:
diesterification of 5-hydroxyisophthalic acid reaction of the hydroxy group with bromo-R₁,
hydrolization of the diester to the diacid,
converting the diacid into the diacid chloride, and
reaction of the diacid chloride with anthranilic acid or its derivatives.

6. A light and heat stabilizer for organic polymers comprising an isobenzoxazinone according to claim 1.

7. A cosmetic formulation comprising an isobenzoxazinone according to claim 1.

8. A process for the stabilisation of an organic polymer comprising the step of incorporating at least one isobenzoxazinone according to claim 1 into the organic polymer in a concentration from 0.005 to 0.100 weight percent based on the weight of the organic polymer.

9. An organic polymer composition comprising at least one isobenzoxazinone according to claim 1 in a concentration from 0.005 to 0.100 weight percent based on the weight of the organic polymer.

10. An organic polymer composition according to claim 9, wherein the organic polymer is selected from the group consisting of polycarbonate, polyester and polyamide.

11. Isobenzoxazinones according to claim 1, wherein the halogen is selected from the group consisting of F, Cl, Br and I.
